(19) United States
(12) Patent Application Publication
(10) Pub. No.: US 2014/0112992 A1
(43) Pub. Date: Apr. 24, 2014

(54) PROCESS FOR FEBUXOSTAT

(71) Applicants: Bandi Parthasaradhi Reddy, Hyderabad (IN); Kura Rathnakar Reddy, Hyderabad (IN); Dasari Muralidhara Reddy, Hyderabad (IN); Matta Ramakrishna Reddy, Hyderabad (IN); Bandi Vamsi Krishna, Hyderabad (IN)

(72) Inventors: Bandi Parthasaradhi Reddy, Hyderabad (IN); Kura Rathnakar Reddy, Hyderabad (IN); Dasari Muralidhara Reddy, Hyderabad (IN); Matta Ramakrishna Reddy, Hyderabad (IN); Bandi Vamsi Krishna, Hyderabad (IN)

(73) Assignee: HETERO RESEARCH FOUNDATION, Hyderabad, Andhrapradesh (IN)

(21) Appl. No.: 14/123,875

(22) PCT Filed: May 21, 2012
(86) PCT No.: PCT/IN12/00359

§ 371 Date: Dec. 4, 2013

(30) Foreign Application Priority Data
Jun. 6, 2011 (IN) 1907/CHE/2011

(51) Int. Cl. C07D 277/56; A61K 31/426

(52) U.S. Cl. CPC C07D 277/56; A61K 31/426
USPC 424/489; 548/201; 514/365

(57) ABSTRACT

The present invention provides a process for the preparation of 2-(4-hydroxyphenyl)-4-methylthiazole-5-carboxylic acid ethyl ester. The present invention also provides a process for the preparation of 2-(3-formyl-4-hydroxyphenyl)-4-methylthiazole-5-carboxylic acid ethyl ester. The present invention further provides novel crystalline Forms of febuxostat, processes for their preparation and pharmaceutical compositions comprising them. The present invention further provides febuxostat crystalline particles having a mean particle size of less than about 25 μm, the methods for the manufacture of said crystalline particles, and pharmaceutical compositions comprising said crystalline particles.

This application claims the benefit of Indian Patent Application No. 1907/CHE/2011, filed on Jun. 6, 2011, which is incorporated herein by reference.

### FIELD OF THE INVENTION

The present invention provides a process for the preparation of 2-(4-hydroxyphenyl)-4-methylthiazole-5-carboxylic acid ethyl ester. The present invention also provides a process for the preparation of 2-(3-formyl-4-hydroxyphenyl)-4-methylthiazole-5-carboxylic acid ethyl ester. The present invention further provides novel crystalline Forms of febuxostat, processes for their preparation and pharmaceutical compositions comprising them. The present invention further provides febuxostat crystalline particles having a mean particle size of less than about 25 μm, the methods for the manufacture of said crystalline particles, and pharmaceutical compositions comprising said crystalline particles.

### BACKGROUND OF THE INVENTION

Febuxostat is chemically, 2-[3-cyano-4-(2-methylpropoxy)phenyl]-4-methyl-5-thiazolecarboxylic acid and has the structural formula:

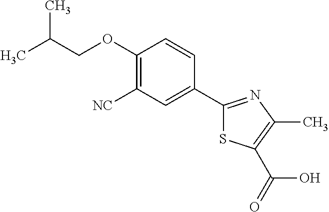 [SMILES: Cc1nc(-c2ccc(OCC(C)C)c(C#N)c2)sc1C(=O)O]

Febuxostat (brand names Adenuric (EU) and Uloric (US)) is an inhibitor of xanthine oxidase that is indicated for use in the treatment of hyperuricemia and gout. The drug is marketed by Menarini. A study comparing febuxostat to allopurinol found that more individuals treated with febuxostat had decreased levels of uric acid, but there was no difference in the amount of initial gout flares or the surface area of gout tophi.

Febuxostat and its process were disclosed in U.S. Pat. No. 5,614,520.

Polymorphism is defined as “the ability of a substance to exist as two or more crystalline phases that have different arrangement and/or conformations of the molecules in the crystal Lattice. Thus, in the strict sense, polymorphs are different crystalline structures of the same pure substance in which the molecules have different arrangements and/or different configurations of the molecules”. Different polymorphs may differ in their physical properties such as melting point, solubility, X-ray diffraction patterns, etc. Although those differences disappear once the compound is dissolved, they can appreciably influence pharmaceutically relevant properties of the solid form, such as handling properties, dissolution rate and stability. Such properties can significantly influence the processing, shelf life, and commercial acceptance of a polymorph. It is therefore important to investigate all solid forms of a drug, including all polymorphic forms, and to determine the stability, dissolution and flow properties of each polymorphic form. Polymorphic forms of a compound can be distinguished in the laboratory by analytical methods such as X-ray diffraction (XRD), Differential Scanning Calorimetry (DSC) and Infrared spectrometry (IR).

Solvent medium and mode of crystallization play very important role in obtaining one polymorphic Form over the other.

Febuxostat can exist in different polymorphic Forms, which may differ from each other in terms of stability, physical properties, spectral data and methods of preparation.

PCT publication no. WO 2010/142653 disclosed a process for the preparation of febuxostat.

PCT publication no. WO 2011/031409 ('409 patent) disclosed a process for the preparation of febuxostat. According to the '409 patent also disclosed one-pot process for preparing of 2-(3-cyano-4-isobutoxyphenyl)-4-methylthiazole-5-carboxylic acid ethyl ester.

According to the '409 patent, febuxostat can be prepared by hydrolyzing the 2-(3-cyano-4-isobutoxyphenyl)-4-methylthiazole-5-carboxylic acid ethyl ester with sodium hydroxide in the presence of absolute ethanol or n-butanol.

U.S. Pat. No. 6,225,474 disclosed crystalline Form A, Form B, Form C, Form D, Form G and amorphous Form of febuxostat.

PCT publication no. WO 2008/067773 disclosed crystalline Form H, Form I and Form J of febuxostat.

Crystalline Form I and Form II of febuxostat were disclosed in Chinese patent publication no. 101139325.

Chinese patent publication no. 101386605 disclosed a crystalline Form K of febuxostat, characterized by an X-ray powder diffraction pattern having peaks expressed as 2θ at about 5.64, 7.80, 11.38, 11.70, 12.54, 12.74, 17.18 and 26.12±0.2 degrees.

Chinese patent publication no. 101412700 disclosed a crystalline Form of febuxostat, characterized by an X-ray powder diffraction pattern having peaks expressed as 2θ at about 5.54, 5.66, 7.82, 11.48, 12.62, 16.74, 17.32, 18.04, 18.34, 20.40, 23.74, 25.76 and 26.04±0.2 degrees.

Crystalline Form Q of febuxostat was disclosed in Chinese patent publication no. 101648926.

Chinese patent publication no. 101671315 disclosed a crystalline Form K of febuxostat, characterized by an X-ray powder diffraction pattern having peaks expressed as 2θ at about 4.82, 6.64, 6.88, 7.22, 11.74, 12.82, 13.28, 16.00, 16.50, 17.50, 20.98, 22.02, 23.00, 23.82, 24.70, 25.18, 25.84 and 26.68±0.2 degrees.

Crystalline Form X, Form Y and Form Z of febuxostat were disclosed in Chinese patent publication no. 101684107.

An unpublished application, IN 2810/CHE/2010 assigned to Hetero research foundation discloses a crystalline Form H1 and Form H2 of febuxostat.

We have found a novel process for the preparation of 2-(4-hydroxyphenyl)-4-methylthiazole-5-carboxylic acid ethyl ester.

We have also found a novel process for the preparation of 2-(3-formyl-4-hydroxyphenyl)-4-methylthiazole-5-carboxylic acid ethyl ester.

We have also found novel crystalline Forms of febuxostat. The novel crystalline Forms have been found to be reproducible and so, suitable for pharmaceutical preparations.

The febuxostat product prepared by the methods as described in the prior art has a very large particle size resulting in similarly poor dissolution properties.

It is known that, particle size can affect the dissolution properties of a drug product. Particle size reduction may be tried in order to increase dissolution characteristics of febuxostat.

Particle size also can affect how freely crystals or a powdered form of a drug will flow past each other, which has consequences in the production process of pharmaceutical products containing the drug.

In view of the foregoing, there is a need in the medical arts for febuxostat with a small particle size and improved bioavailability.

It has now been determined that compositions comprising crystalline particles of febuxostat having a mean particle size equal to or less than about 25 μm exhibit good dissolution properties.

Thus, one object of the present invention is to provide a novel process for the preparation of 2-(4-hydroxyphenyl)-4-methylthiazole-5-carboxylic acid ethyl ester.

Another object of the present invention is to provide a novel process for the preparation of 2-(3-formyl-4-hydroxyphenyl)-4-methylthiazole-5-carboxylic acid ethyl ester.

Another object of the present invention is to provide novel crystalline Forms of febuxostat, processes for their preparation and pharmaceutical compositions comprising them.

Another object of the present invention is to provide febuxostat crystalline particles and formulations containing febuxostat crystalline particles having a mean particle size of less than about 25 μm, and methods for manufacturing such particles.

### SUMMARY OF THE INVENTION

In one aspect, the present invention provides a novel process for the preparation of 2-(4-hydroxyphenyl)-4-methylthiazole-5-carboxylic acid ethyl ester, which comprises reacting the 4-hydroxythiobenzamide with 2-chloroacetoacetic acid ethyl ester in the presence of an alcoholic solvent.

In another aspect, the present invention provides a novel process for the preparation of 2-(3-formyl-4-hydroxyphenyl)-4-methylthiazole-5-carboxylic acid ethyl ester, which comprise:
a) reacting the 2-(4-hydroxyphenyl)-4-methylthiazole-5-carboxylic acid ethyl ester with trifluoroacetic acid in the presence of hexamethyl tetramine;
b) heating the reaction mixture obtained in step (a) at above 60° C.;
c) cooling the reaction mass obtained in step (b) at below 35° C.;
d) extracting the reaction mass into toluene;
e) removing the solvent from the reaction mass obtained in step (d) to obtain a residual solid;
f) slurring the residual solid obtained in step (e) with cyclohexane; and
g) isolating the 2-(3-formyl-4-hydroxyphenyl)-4-methylthiazole-5-carboxylic acid ethyl ester.

In another aspect, the present invention provides a novel crystalline form of febuxostat designated as Form H3 characterized by peaks in the powder x-ray diffraction spectrum having 2θ angle positions at about 4.8, 5.5, 6.0, 11.0, 11.3, 11.8, 12.1, 15.6, 16.6, 16.9, 25.2, 25.7 and 26.2±0.2 degrees.

In another aspect, the present invention provides a process for the preparation of febuxostat crystalline Form H3, which comprises:
a) suspending febuxostat in cyclohexane;
b) heating the suspension obtained in step (a) at above 55° C.;
c) cooling the reaction mass obtained in step (b) at below 20° C.; and
d) isolating febuxostat crystalline Form H3.

In another aspect, the present invention provides a pharmaceutical composition comprising crystalline Form H3 of febuxostat and pharmaceutically acceptable excipients.

In another aspect, the present invention provides a crystalline form of febuxostat designated as Form H4 characterized by peaks in the powder x-ray diffraction spectrum having 2θ angle positions at about 4.8, 5.6, 5.8, 6.6, 6.8, 7.2, 8.0, 11.6, 12.8 and 25.9±0.2 degrees.

In another aspect, the present invention provides a process for the preparation of febuxostat crystalline Form H4, which comprises:
a) dissolving febuxostat in an ester solvent by using above 16 volumes with respect to febuxostat;
b) heating the solution obtained in step (a) at reflux;
c) cooling the solution at below 20° C.; and
d) isolating febuxostat crystalline Form H4.

In another aspect, the present invention provides a pharmaceutical composition comprising crystalline Form H4 of febuxostat and pharmaceutically acceptable excipients.

In another aspect, the present invention provides crystalline particles of febuxostat having a mean particle size of less than about 25 μm.

In another aspect, the present invention provides a process for the preparation of crystalline particles of febuxostat having a mean particle size of less than about 25 μm, which comprises grinding the febuxostat.

Yet in another aspect, the present invention provides a pharmaceutical composition comprising febuxostat crystalline particles having a mean particle size of less than about 25 μm and pharmaceutically acceptable excipients.

### BRIEF DESCRIPTION OF THE DRAWINGS

FIG. 1 is an X-ray powder diffraction spectrum of febuxostat crystalline Form H3.

FIG. 2 is an X-ray powder diffraction spectrum of febuxostat crystalline Form H4.

X-ray powder diffraction spectrum was measured on a bruker axs D8 advance X-ray powder diffractometer having a copper-Kα radiation. Approximately 500 mg of sample was gently flattered on a sample holder and scanned from 2 to 50 degrees two-theta, at 0.020 degrees two theta per step and a step time of 1 second. The sample was simply placed on the sample holder. The sample was rotated at 30 rpm at a voltage 40 KV and current 35 mA.

### DETAILED DESCRIPTION OF THE INVENTION

The term “room temperature” refers to temperature at about 25 to 35° C.

According to another aspect of the present invention, there is provided a novel process for the preparation of 2-(4-hydroxyphenyl)-4-methylthiazole-5-carboxylic acid ethyl ester, which comprises reacting the 4-hydroxythiobenzamide with 2-chloroacetoacetic acid ethyl ester in the presence of an alcoholic solvent.

The alcoholic solvent used in the process may preferably be a solvent or mixture of solvents selected from methanol, ethanol, isopropanol and n-butanol, and more preferably the alcoholic solvent is isopropanol.

According to another aspect of the present invention, there is provided a novel process for the preparation of 2-(3-formyl-4-hydroxyphenyl)-4-methylthiazole-5-carboxylic acid ethyl ester, which comprise:
a) reacting the 2-(4-hydroxyphenyl)-4-methylthiazole-5-carboxylic acid ethyl ester with trifluoroacetic acid in the presence of hexamethyl tetramine;
b) heating the reaction mixture obtained in step (a) at above 60° C.;
c) cooling the reaction mass obtained in step (b) at below 35° C.;
d) extracting the reaction mass into toluene;
e) removing the solvent from the reaction mass obtained in step (d) to obtain a residual solid;
f) slurring the residual solid obtained in step (e) with cyclohexane; and
g) isolating the 2-(3-formyl-4-hydroxyphenyl)-4-methylthiazole-5-carboxylic acid ethyl ester.

The reaction in step (b) may preferably be carried out at about 75 to 85° C.

The reaction in step (c) may preferably be carried out at about 20 to 30° C.

Removal of the solvent may be carried out in step (e) at atmospheric pressure or at reduced pressure. Removal of the solvent may preferably be carried out until the solvent is almost completely distilled off.

The isolation of 2-(3-formyl-4-hydroxyphenyl)-4-methylthiazole-5-carboxylic acid ethyl ester in step (g) may be carried out by the methods known such as filtration or centrifugation.

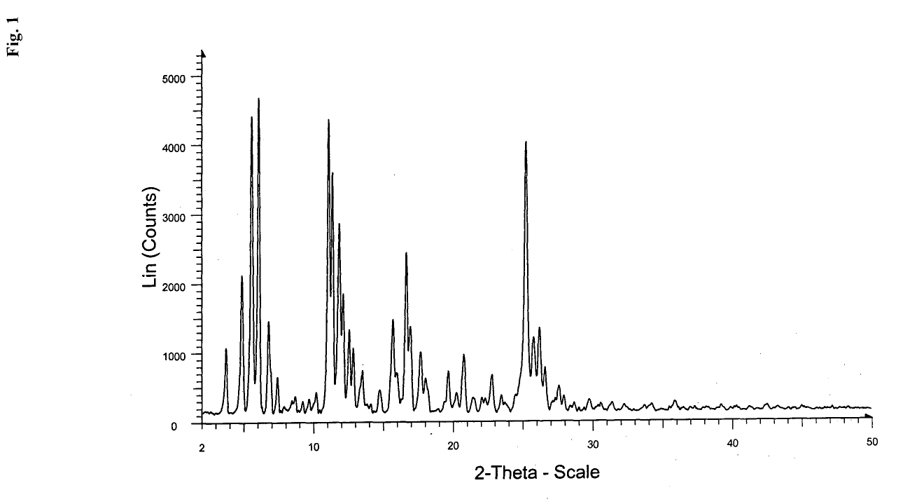

According to another aspect of the present invention, there is provided a novel crystalline form of febuxostat designated as Form H3 characterized by peaks in the powder x-ray diffraction spectrum having 2θ angle positions at about 4.8, 5.5, 6.0, 11.0, 11.3, 11.8, 12.1, 15.6, 16.6, 16.9, 25.2, 25.7 and 26.2±0.2 degrees. The powdered x-ray diffractogram (PXRD) of febuxostat crystalline Form H3 is shown in FIG. 1.

The febuxostat crystalline Form H3 of the present invention may also useful for preparation of febuxostat crystalline Form H1 or other polymorphs of febuxostat.

According to another aspect of the present invention, there is provided a process for the preparation of febuxostat crystalline Form H3, which comprises:
a) suspending febuxostat in cyclohexane;
b) heating the suspension obtained in step (a) at above 55° C.;
c) cooling the reaction mass obtained in step (b) at below 20° C.; and
d) isolating febuxostat crystalline Form H3.

Febuxostat used in step (a) may be any known crystalline or amorphous Forms.

The reaction in step (b) may preferably be carried out at about 60 to 70° C.

The reaction mass may preferably be cooled in step (c) at about 0 to 10° C.

Febuxostat crystalline Form H3 may be isolated in step (d) by methods known such as filtration or centrifugation.

According to another aspect of the present invention, there is provided a pharmaceutical composition comprising crystalline Form H3 of febuxostat and pharmaceutically acceptable excipients, and optionally other therapeutic ingredients. The crystalline Form H3 may preferably be formulated into tablets, capsules, suspensions, dispersions, injectables or other pharmaceutical forms.

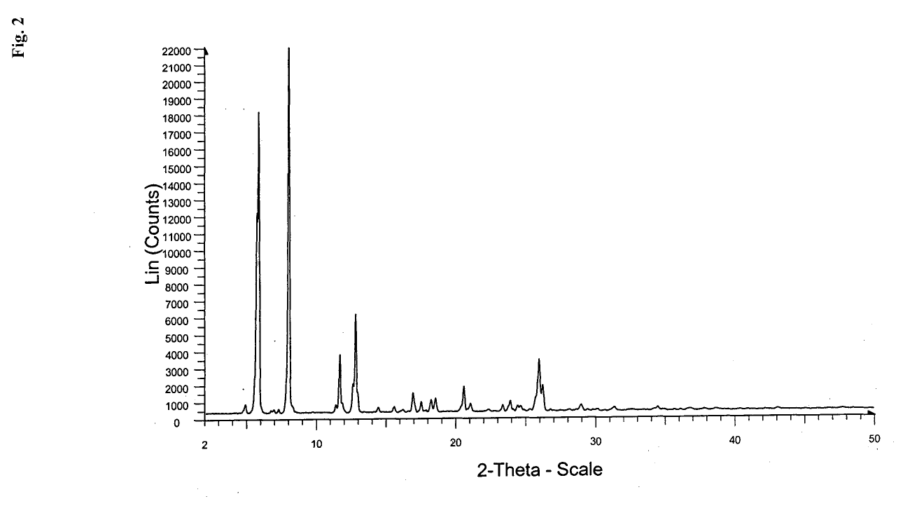

According to another aspect of the present invention, there is provided a crystalline form of febuxostat designated as form H4 characterized by peaks in the powder x-ray diffraction spectrum having 2θ angle positions at about 4.8, 5.6, 5.8, 6.6, 6.8, 7.2, 8.0, 11.6, 12.8 and 25.9±0.2 degrees. The powdered x-ray diffractogram (PXRD) of febuxostat crystalline form H4 is shown in FIG. 2.

The febuxostat crystalline Form H4 may be identified and differentiated from the known polymorphs by its characteristic PXRD pattern. Thus, for example, peaks at 5.8 and 6.6 degrees 2θ are present in the PXRD of the febuxostat crystalline Form H4 of the present invention, but are absent in the PXRD of the crystalline Form K of febuxostat disclosed in the Chinese patent publication no. 101386605. Similarly, peaks at 4.8, 6.6, 6.8 and 7.2 degrees 2θ are present in the PXRD of the febuxostat crystalline Form H4 of the present invention, but are absent in the PXRD of the crystalline Form of febuxostat disclosed in the Chinese patent publication no. 101412700.

According to another aspect of the present invention, there is provided a process for the preparation of febuxostat crystalline Form H4, which comprises:
a) dissolving febuxostat in an ester solvent by using above 16 volumes with respect to febuxostat;
b) heating the solution obtained in step (a) at reflux;
c) cooling the solution at below 20° C.; and
d) isolating febuxostat crystalline Form H4.

Febuxostat used in step (a) may be any known crystalline or amorphous Forms.

The ester solvent used in step (a) may preferably be a solvent or mixture of solvents selected from ethyl acetate, methyl acetate, isopropyl acetate, tert-butyl acetate and ethyl formate, and more preferably the ester solvent is ethyl acetate.

The step (c) may preferably be carried out at about 0 to 5° C.

Febuxostat crystalline Form H4 may be isolated in step (d) by methods known such as filtration or centrifugation.

According to another aspect of the present invention, there is provided a pharmaceutical composition comprising crystalline Form H4 of febuxostat and pharmaceutically acceptable excipients, and optionally other therapeutic ingredients. The crystalline Form H4 may preferable be formulated into tablets, capsules, suspensions, dispersions, injectables and other pharmaceutical forms.

According to another aspect of the present invention, there is provided crystalline particles of febuxostat having a mean particle size of less than about 25 μm.

Preferably, the mean particle size distribution of crystalline particles of febuxostat of ranges from about 0 to 20 μm, and more preferably from about 2 to 15 μm.

According to another aspect of the present invention, there is provided a process for the preparation of crystalline particles of febuxostat having a mean particle size of less than about 25 μm, which comprises grinding the febuxostat.

According to another aspect of the present invention, there is provided a pharmaceutical composition comprising febuxostat crystalline particles having a mean particle size of less than about 25 μm and pharmaceutically acceptable excipients, and optionally other therapeutic ingredients. The crystalline particles may preferably be formulated into tablets, capsules, suspensions, dispersions, injectables or other pharmaceutical forms.

Preferably, the mean particle size distribution of crystalline particles of febuxostat of ranges from about 0 to 20 μm, and more preferably from about 2 to 15 μm. The crystalline particles of febuxostat are used in pharmaceutical compositions.

Febuxostat used in the process may preferably be any known crystalline Forms.

Unless otherwise indicated, the following definitions are set forth to illustrate and define the meaning and scope of the various terms used to describe the invention herein.

The term “μm” refers to “micrometer” which is 1×10⁻⁶ meter.

The term “crystalline particles” means any combination of single crystals, aggregates and agglomerates.

The term “Particle Size Distribution (P.S.D.)” means the cumulative volume size distribution of equivalent spherical diameters as determined by laser diffraction at 1 bar dispersive pressure in Sympatec Helos equipment. “Mean particle size distribution, i.e., D₅₀” correspondingly, means the median of said particle size distribution.

The invention will now be further described by the following example, which is illustrative rather than limiting.

### EXAMPLES

### Example 1

### Preparation of 2-(4-hydroxyphenyl)-4-methylthiazole-5-carboxylic acid ethyl ester

4-Hydroxythiobenzamide (250 gm) was added to isoprapanol (1250 ml) at room temperature and then heated to 50° C. To the solution was added 2-chloroacetoacetic acid ethyl ester (300 gm) slowly for 30 minutes at 50 to 55° C. and then heated to 80 to 85° C. The reaction mass was then cooled to room temperature and stirred for 1 hour. The contents were further cooled to 10 to 15° C. and filtered. The solid obtained was dried to give 415 gm of 2-(4-hydroxyphenyl)-4-methylthiazole-5-carboxylic acid ethyl ester.

### Example 2

### Preparation of 2-(3-formyl-4-hydroxyphenyl)-4-methylthiazole-5-carboxylic acid ethyl ester

2-(4-Hydroxyphenyl)-4-methylthiazole-5-carboxylic acid ethyl ester (413 gm) was added to trifluoroacetic acid (2065) and then added hexamethyl tetramine (248 gm) at room temperature. The contents were heated to 80° C. and maintained for 24 hours. The reaction mass was then cooled to room temperature and mass was poured into child water (5155 ml). The reaction mass was extracted with toluene (2887 ml) and stirred for 10 minutes. Then the layers were separated and the aqueous layer was extracted with toluene. Combined organic layers were dried with sodium sulfate and then concentrated to obtain a residual solid. To the residual solid was added cyclohexane (1250 ml) at room temperature and then heated to reflux for 30 minutes. The reaction mass was then cooled to room temperature and stirred for 1 hour. The solid obtained was collected by filtration and dried to obtain 260 gm of 2-(3-formyl-4-hydroxyphenyl)-4-methylthiazole-5-carboxylic acid ethyl ester.

### Example 3

### Preparation of 2-(3-formyl-4-isobutoxyphenyl)-4-methylthiazole-5-carboxylic acid ethyl ester

To a dimethylformamide (1250 ml) was added 2-(3-formyl-4-hydroxyphenyl)-4-methylthiazole-5-carboxylic acid ethyl ester (250 gm) at room temperature for 5 minutes to obtain a solution. To the solution was added potassium carbonate (250 gm) and potassium iodide (62.5 gm), and then added isobutyl bromide (100 gm) slowly for 2 hours. The temperature of the reaction mass was raised to 85° C. and maintained for 3 hours. The dimethylformamide solvent was distilled off under vacuum at 80° C. and then cooled to room temperature. The reaction mass was added water (2500 ml) and stirred for 2 hours at room temperature. The separated solid was filtered, washed with cyclohexane and then dried to obtain 285 gm of 2-(3-formyl-4-isobutoxyphenyl)-4-methylthiazole-5-carboxylic acid ethyl ester.

### Example 4

### Preparation of 2-(3-cyano-4-isobutoxyphenyl)-4-methylthiazole-5-carboxylic acid ethyl ester

To a 2-(3-formyl-4-isobutoxyphenyl)-4-methylthiazole-5-carboxylic acid ethyl ester (285 gm) was added formic acid (713 ml) and then added hydroxylamine (71 gm) at room temperature to obtain a solution. To the solution was added sodium formate (86 gm) and stirred for 10 minutes. The temperature of the reaction mass was raised to 100° C. and maintained for 2 hours. The solvent was distilled off under vacuum and the mass was then cooled to room temperature, and then added water (2850 ml). The solid obtained was collected by filtration and washed with toluene and then dried to obtain 243 gm of 2-(3-cyano-4-isobutoxyphenyl)-4-methylthiazole-5-carboxylic acid ethyl ester.

### Example 5

### Preparation of Febuxostat

2-(3-Cyano-4-isobutoxyphenyl)-4-methylthiazole-5-carboxylic acid ethyl ester (100 gm) was dissolved in ethanol (500 ml) at room temperature and then added a solution of sodium hydroxide (13 gm) in water. (250 ml) slowly for 30 minutes. The contents were heated to 65° C. and maintained for 1 hour. The reaction mass was then cooled to 50° C. and pH of the reaction mass was adjusted to 2.0 with hydrochloric acid solution (15%). The reaction mass was stirred for 15 minutes at 50° C. and then cooled to room temperature. The contents were maintained for 3 hours at room temperature and filtered. The solid thus obtained was dissolved in ethanol (500 ml) and then heated to 60° C. The reaction mass was treated with carbon at 60° C. and filtered. The filtrate obtained was then cooled to room temperature and stirred for 2 hours. The reaction mass was further cooled to 10° C. and maintained for 1 hour. The solid obtained was collected by filtration and washed with chilled ethanol to obtain a wet solid. To the wet solid was added a mixture of ethanol and water (9:1; 500 ml) at room temperature and then heated to 60° C. to obtain a solution. The solution was stirred for 30 minutes at 60° C. and then cooled to 25° C. The reaction mass was maintained for 1 hour at 25° C. and the mass was further cooled to 10° C. The solid obtained was collected by filtration and dried with vacuum sucking for 15 minutes to obtain 65 gm of febuxostat.

### Preparation of Febuxostat Crystalline Form H3

### Example 6

Febuxostat (65 gm) as obtained in example 5 was suspended in cyclohexane (1000 ml) and then heated to 60° C. The reaction mass was stirred for 1 hour at 60° C. and then cooled to room temperature. The reaction mass was maintained for 1 hour at room temperature and filtered. The solid obtained was dried to give 63 gm of febuxostat crystalline Form H3.

### Example 7

Febuxostat crystalline Form H1 (50 gm) was suspended in cyclohexane (760 ml) and then heated to 60° C. The reaction mass was stirred for 1 hour at 60° C. and then cooled to room temperature. The reaction mass was maintained for 1 hour at room temperature and filtered. The solid obtained was dried to give 48 gm of febuxostat crystalline Form H3.

### Example 8

Example 7 was repeated using febuxostat crystalline Form H2 instead of febuxostat crystalline Form H1 to obtain febuxostat crystalline Form H3.

### Example 9

Example 7 was repeated using febuxostat crystalline Form G instead of febuxostat crystalline Form H1 to obtain febuxostat crystalline Form H3.

### Example 10

Example 7 was repeated using febuxostat crystalline Form C instead of febuxostat crystalline Form H1 to obtain febuxostat crystalline Form H3.

### Preparation of Febuxostat Crystalline Form H1

### Example 11

Febuxostat crystalline Form H3 (65 gm) as obtained in example 6 was dissolved in ethyl acetate (1200 ml) and then heated to reflux for 10 minutes to obtain a solution. The solution was then cooled to 0 to 5° C. and maintained for 1 hour. The solid obtained was collected by filtration and dried under vacuum at 45° C. for 12 hours to obtain 56 gm of febuxostat crystalline Form H1.

### Example 12

Febuxostat crystalline Form H1 (10 gm; D₅₀: 322.46) was grinded for 5 to 10 minutes to obtain febuxostat crystalline Form H1 (D₅₀: 2.15 μm).

### Preparation of Febuxostat Crystalline Form H4

### Example 13

Febuxostat (5 Kg) as obtained by the process of example 5 was dissolved in ethyl acetate (90 L) and then heated to reflux. The reaction mass was stirred for 30 minutes at reflux to a clear solution. To the solution was then cooled to 0 to 5° C. for 1 hour 15 minutes and then maintained for 1 hour 30 minutes at 0 to 5° C. The separated solid was filtered and then dried to give 4.2 Kg of febuxostat crystalline Form H4.

### Example 14

Febuxostat (50 gm) was dissolved in ethyl acetate (900 ml) and then heated to reflux. The reaction mass was stirred for 30 minutes at reflux to a clear solution. To the solution was then cooled to 0 to 5° C. for 45 minutes and then maintained for 1 hour 30 minutes at 0 to 5° C. The separated solid was filtered and then dried to give 42 gm of febuxostat crystalline Form H4.

### Example 15

Febuxostat crystalline Form H1 (5 gm) was dissolved in ethyl acetate (100 ml). The reaction mass was then heated to reflux and stirred for 1 hour at reflux to obtain a clear solution. To the solution was then cooled to 0 to 5° C. for 45 minutes and then maintained for 1 hour 30 minutes at 0 to 5° C. The separated solid was filtered and then dried to give 4 gm of febuxostat crystalline Form H4.

### Example 16

Example 15 was repeated using febuxostat crystalline Form H2 instead of febuxostat crystalline Form H1 to obtain febuxostat crystalline Form H4.

### Example 17

Example 15 was repeated using febuxostat crystalline Form G instead of febuxostat crystalline Form H1 to obtain febuxostat crystalline Form H4.

### Example 18

Example 15 was repeated using febuxostat crystalline Form C instead of febuxostat crystalline Form H1 to obtain febuxostat crystalline Form H4.

### Example 19

Example 13 was repeated using isopropyl acetate solvent instead of ethyl acetate solvent to obtain febuxostat crystalline Form H4.

### Example 20

Example 13 was repeated using tert-butyl acetate solvent instead of ethyl acetate solvent to obtain febuxostat crystalline Form H4.

1. A process for the preparation of 2-(4-hydroxyphenyl)-4-methylthiazole-5-carboxylic acid ethyl ester, which comprises reacting the 4-hydroxythiobenzamide with 2-chloroacetoacetic acid ethyl ester in the presence of an alcoholic solvent.

2. The process according to claim 1, wherein the alcoholic solvent is a solvent or mixture of solvents selected from methanol, ethanol, isopropanol and n-butanol.

3. The process according to claim 2, wherein the alcoholic solvent is isopropanol.

4. A process for the preparation of 2-(3-formyl-4-hydroxyphenyl)-4-methylthiazole-5-carboxylic acid ethyl ester, which comprise:
a. reacting the 2-(4-hydroxyphenyl)-4-methylthiazole-5-carboxylic acid ethyl ester with trifluoroacetic acid in the presence of hexamethyl tetramine;
b. heating the reaction mixture obtained in step (a) at above 60° C.;
c. cooling the reaction mass obtained in step (b) at below 35° C.;
d. extracting the reaction mass into toluene;
e. removing the solvent from the reaction mass obtained in step (d) to obtain a residual solid;
f. slurring the residual solid obtained in step (e) with cyclohexane; and
g. isolating the 2-(3-formyl-4-hydroxyphenyl)-4-methylthiazole-5-carboxylic acid ethyl ester.

5. The process according to claim 4, wherein the reaction in step (b) is carried out at about 75 to 85° C.

6. The process according to claim 4, wherein the reaction in step (c) is carried out at about 20 to 30° C.

7. A febuxostat crystalline Form H3 which is characterized by peaks in the powder x-ray diffraction spectrum having 2θ angle positions at about 4.8, 5.5, 6.0, 11.0, 11.3, 11.8, 12.1, 15.6, 16.6, 16.9, 25.2, 25.7 and 26.2±0.2 degrees.

8. A febuxostat crystalline Form H3 which is characterized by an X-Ray Powder Diffractogram as shown in FIG. 1.

9. A process for the preparation of febuxostat crystalline Form H3 as claimed in claim 7, which comprises:
a. suspending febuxostat in cyclohexane;
b. heating the suspension obtained in step (a) at above 55° C.;
c. cooling the reaction mass obtained in step (b) at below 20° C.; and
d. isolating febuxostat crystalline Form H3.

10. The process according to claim 9, wherein the reaction in step (b) is carried out at about 60 to 70° C.

11. The process according to claim 9, wherein the reaction mass is cooled in step (c) at about 0 to 10° C.

12. A febuxostat crystalline Form H4 which is characterized by peaks in the powder x-ray diffraction spectrum having 2θ angle positions at about 4.8, 5.6, 5.8, 6.6, 6.8, 7.2, 8.0, 11.6, 12.8 and 25.9±0.2 degrees.

13. A febuxostat crystalline Form H4 which is characterized by an X-Ray Powder Diffractogram as shown in FIG. 2.

14. A process for the preparation of febuxostat crystalline Form H4 as claimed in claim 12, which comprises:
a. dissolving febuxostat in an ester solvent by using above 16 volumes with respect to febuxostat;
b. heating the solution obtained in step (a) at reflux;
c. cooling the solution at below 20° C.; and
d. isolating febuxostat crystalline Form H4.

15. The process according to claim 14, wherein the ester solvent used in step (a) is a solvent or mixture of solvents selected from ethyl acetate, methyl acetate, isopropyl acetate, tert-butyl acetate and ethyl formate.

16. The process according to claim 15, wherein the ester solvent is ethyl acetate.

17. The process according to claim 14, wherein the step (c) is carried out at about 0 to 5° C.

18. Crystalline particles of febuxostat having a mean particle size of less than about 25 μm.

19. The crystalline particles of febuxostat as claimed in claim 18, wherein the mean particle size ranges from about 0 to 20 μm.

20. The crystalline particles of febuxostat as claimed in claim 19, wherein the mean particle size ranges from about 2 to 15 μm.

21. A process for the preparation of crystalline particles of febuxostat having a mean particle size of less than about 25 μm, which comprises grinding the febuxostat.

22. A pharmaceutical composition that comprises crystalline Form H3 of febuxostat and pharmaceutically acceptable excipients, and optionally other therapeutic ingredients.

23. A pharmaceutical composition that comprises crystalline Form H4 of febuxostat and pharmaceutically acceptable excipients, and optionally other therapeutic ingredients.

24. A pharmaceutical composition comprising febuxostat crystalline particles having a mean particle size of less than about 25 μm and pharmaceutically acceptable excipients, and optionally other therapeutic ingredients.

25. The pharmaceutical composition as claimed in claim 22, wherein the polymorphic forms are formulated into tablets, capsules, suspensions, dispersions or injectables.

26. The pharmaceutical composition as claimed in claim 23, wherein the polymorphic forms are formulated into tablets, capsules, suspensions, dispersions or injectables.

27. The pharmaceutical composition as claimed in claim 24, wherein the polymorphic forms are formulated into tablets, capsules, suspensions, dispersions or injectables.
